(19) United States
(12) Patent Application Publication
(10) Pub. No.: US 2010/0168705 A1
(43) Pub. Date: Jul. 1, 2010

(54) Disposable Absorbent Garments Employing Elastomeric Film Laminates With Deactivated Regions

(76) Inventors: Sara J. Stabelfeldt, Appleton, WI (US); Stephen Carl Baumgartner, Neenah, WI (US); Eric-John Raoul Gilgenbach, Winneconne, WI (US); Suzanne M. Schmoker, Oshkosh, WI (US); Frederique Vignali, Appleton, WI (US)

Correspondence Address: KIMBERLY-CLARK WORLDWIDE, INC.;Tara Pohlkotte, 2300 Winchester Rd., NEENAH, WI, 54956, US

(21) Appl. No.: 12/346,060

(22) Filed: Dec. 30, 2008

(51) Int. Cl. A61F 13/15

(52) U.S. Cl. 604/367; 604/385.23; 604/385.29

(57) ABSTRACT

An absorbent garment comprises an absorbent member and a laminate. The laminate comprises a polymeric film layer and a nonwoven layer, wherein both the polymeric film layer and the nonwoven layer extend substantially throughout the entire area of the laminate. At least a portion of the absorbent member longitudinally overlaps at least a portion of the laminate to define in the laminate an overlapped region and a non-overlapped region. At least a portion of the overlapped region of the laminate is non-elastomeric, and at least a portion of the non-overlapped region of the laminate is elastomeric. In particular embodiments, the laminate is an elastomeric film laminate having an elastomeric film layer, and at least a portion of the overlapped region of the elastomeric film laminate is deactivated.

### BACKGROUND

People rely on disposable absorbent products in their everyday lives, including such articles as adult incontinence products, enuresis pants, training pants, and diapers. Many manufacturers seek to better meet the needs of users of such products. For example, there is a need to further improve fit, discretion, and leakage protection for many products.

Some products employ elasticization across the front and/or back of the garment to assist in keeping the product fit snugly against the wearer. Some products employ a multiplicity of elastic strands within front and/or back waist panels to provide the elasticization, while other products employ elastomeric polymeric films, often sandwiched with one or more nonwoven layers. Most products also include an absorbent member, constructed from wood pulp fluff, superabsorbent polymers, or other absorbent material to absorb fluids such as urine. The absorbent member is typically positioned in the crotch region and extends forward and backward into the front and/or back regions of the product. In certain prior art products, some of the elastic members that extend across the front and/or back waist panels overlap the absorbent member at various locations, by virtue of the absorbent member extending forward/backward into the front/back waist regions. This can be undesirable, because the tension of the elastic members can tend to gather the absorbent member, or cause it to “bunch.” Such bunching of the absorbent member can create fit and discretion problems. From a fit standpoint, a bunched absorbent is less likely to lie snugly against the body, potentially increasing the incidence of leakage. From a discretion standpoint, excessive bunching tends to make the product more bulky and therefore more visible under clothing. This circumstance is particularly problematic for incontinence articles, such as enuresis pants and adult pull-on style disposable absorbent underwear, as the wearers of such products generally are embarrassed about their condition and wish to employ protection which is as discreet as possible.

Therefore, there remains a need for absorbent garments having elasticized panels that are less likely to cause undesirable gathering and bunching of the absorbent member. There also remains a need for a process of manufacturing such articles.

### SUMMARY OF THE INVENTION

In response to the aforementioned needs, a new absorbent garment and a process for making such a garment have been invented. In one aspect, the present invention relates to an absorbent garment. In another aspect, the present invention relates to a process for manufacturing absorbent garments.

In one embodiment of the garment aspect of the present invention, an absorbent garment defines a longitudinal direction and a transverse direction. The absorbent garment comprises a front region defining a front edge and first and second transversely opposed front side edges; a back region defining a back edge and first and second transversely opposed back side edges; a crotch region disposed longitudinally between and interconnecting the front and back regions; and an absorbent member extending longitudinally from the front region to the back region. The front region comprises a laminate that comprises a polymeric film layer and a nonwoven layer, wherein both the polymeric film layer and the nonwoven layer extend substantially throughout the entire area of the laminate. At least a portion of the absorbent member longitudinally overlaps at least a portion of the laminate to define in the laminate an overlapped region and a non-overlapped region. At least a portion of the overlapped region of the laminate is non-elastomeric, and wherein at least a portion of the non-overlapped region of the laminate is elastomeric.

In another embodiment of the garment aspect of the present invention, an absorbent garment defines a longitudinal direction and a transverse direction. The absorbent garment comprises a front region defining a front edge and first and second transversely opposed front side edges; a back region defining a back edge and first and second transversely opposed back side edges; a crotch region disposed longitudinally between and interconnecting the front and back regions; and an absorbent member extending longitudinally from the front region to the back region. The front region comprises a polymeric film transversely extending from the first front side edge to the second front side edge. At least a portion of the absorbent member overlaps at least a portion of the film to define in the film an overlapped region and a non-overlapped region. At least a portion of the overlapped region of the polymeric film constitutes a non-elastomeric film, and at least a portion of the non-overlapped region of the polymeric film constitutes an elastomeric film.

In yet another embodiment of the garment aspect of the present invention, an absorbent garment defines a longitudinal direction and a transverse direction. The absorbent garment comprises a front region defining a front edge and first and second transversely opposed front side edges; a back region defining a back edge and first and second transversely opposed back side edges; a crotch region disposed longitudinally between and interconnecting the front and back regions; and an absorbent member extending longitudinally from the front region to the back region. The front region comprises an elastomeric film laminate that comprises an elastomeric film layer and a nonwoven layer, and both the elastomeric film layer and the nonwoven layer extend substantially throughout the entire area of the laminate. At least a portion of the absorbent member longitudinally overlaps at least a portion of the laminate to define in the laminate an overlapped region and a non-overlapped region. At least a portion of the overlapped region of the elastomeric film laminate is deactivated, and at least a portion of the non-overlapped region of the elastomeric film laminate is elastomeric.

### BRIEF DESCRIPTION OF THE DRAWINGS

FIG. 1 representatively illustrates a plan view of one embodiment of an absorbent garment incorporating the principles of the present invention in a longitudinally stretched and laid-flat condition, prior to the joining of the front and back waist regions, and showing the surface of the article that faces the wearer when the article is worn.

FIG. 2 representatively illustrates a plan view of an absorbent insert suitable for use in conjunction with the present invention, with portions cut away to show underlying features.

FIG. 3 representatively illustrates a cross-sectional view taken along line 3-3 in FIG. 2.

FIG. 4 representatively illustrates a cross-sectional view taken along line 4-4 in FIG. 1.

FIG. 5 representatively illustrates a cross-sectional view taken along line 5-5 in FIG. 1.

FIG. 6 representatively illustrates a plan view of an alternative embodiment of an absorbent garment incorporating the principles of the present invention in a longitudinally stretched and laid-flat condition, prior to the joining of the front and back waist regions, and showing the surface of the article that faces the wearer when the article is worn.

FIG. 7 representatively illustrates a plan view of an alternative embodiment of an absorbent garment incorporating the principles of the present invention in a longitudinally stretched and laid-flat condition, prior to the joining of the front and back waist regions, and showing the surface of the article that faces the wearer when the article is worn.

FIG. 8 representatively illustrates a front perspective view of the embodiment of FIG. 7, with the front and back waist regions being joined such that the garment is in a pull-on, pant-like configuration.

FIG. 9 representatively illustrates a plan view of an alternative embodiment of an absorbent garment incorporating the principles of the present invention in a longitudinally stretched and laid-flat condition, prior to the joining of the front and back waist regions, showing the surface of the article that faces the wearer when the article is worn, but without an absorbent insert.

FIG. 10 representatively illustrates a plan view of an alternative embodiment of an absorbent garment incorporating the principles of the present invention in a longitudinally stretched and laid-flat condition, prior to the joining of the front and back waist regions, and showing the surface of the article that faces the wearer when the article is worn.

FIG. 11 representatively illustrates a plan view of an alternative embodiment of an absorbent garment incorporating the principles of the present invention in a longitudinally stretched and laid-flat condition, prior to the joining of the front and back waist regions, and showing the surface of the article that faces the wearer when the article is worn.

FIG. 12 representatively illustrates a plan view of an alternative embodiment of an absorbent garment incorporating the principles of the present invention in a longitudinally stretched and laid-flat condition, prior to the joining of the front and back waist regions, and showing the surface of the article that faces the wearer when the article is worn.

FIG. 13 representatively illustrates a plan view of an alternative embodiment of an absorbent garment incorporating the principles of the present invention in a longitudinally stretched and laid-flat condition, prior to the joining of the front and back waist regions, and showing the surface of the article that faces the wearer when the article is worn.

FIG. 14 representatively illustrates a plan view of an alternative embodiment of an absorbent garment incorporating the principles of the present invention in a longitudinally stretched and laid-flat condition, prior to the joining of the front and back waist regions, and showing the surface of the article that faces the wearer when the article is worn.

FIG. 15 representatively illustrates a plan view of an alternative embodiment of an absorbent garment incorporating the principles of the present invention in a longitudinally stretched and laid-flat condition, prior to the joining of the front and back waist regions, and showing the surface of the article that faces the wearer when the article is worn.

FIG. 16 representatively illustrates a plan view of an alternative embodiment of an absorbent garment incorporating the principles of the present invention in a longitudinally stretched and laid-flat condition, prior to the joining of the front and back waist regions, and showing the surface of the article that faces the wearer when the article is worn.

FIG. 17 representatively illustrates a perspective view of one embodiment of a manufacturing process incorporating the principles of the process aspect of the present invention.

FIG. 18 representatively illustrates a perspective view of an alternative embodiment of a manufacturing process incorporating the principles of the process aspect of the present invention.

FIGS. 19 through 25 representatively illustrate plan views of garment front panels suitable for use with particular embodiments of the present invention.

### DETAILED DESCRIPTION OF PARTICULAR EMBODIMENTS

As used herein, the following terms have the following meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Fiber” refers to a continuous or discontinuous member having a high ratio of length to diameter or width. Thus, a fiber may be a filament, a thread, a strand, a yarn, or any other member or combination of these members.

“Layer” when used in the singular can have the dual meaning of a single element or a plurality of elements.

“Liquid impermeable,” when used in describing a layer or multi-layer laminate means that liquid, such as urine, will not pass through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblowing processes can be used to make fibers of various dimensions, including macrofibers (with average diameters from about 40 to about 100 microns), textile-type fibers (with average diameters between about 10 and 40 microns), and microfibers (with average diameters less than about 10 microns). Meltblowing processes are particularly suited to making microfibers, including ultra-fine microfibers (with an average diameter of about 3 microns or less). A description of an exemplary process of making ultra-fine microfibers may be found in, for example, U.S. Pat. No. 5,213,881 to Timmons, et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

These terms may be defined with additional language elsewhere in the specification.

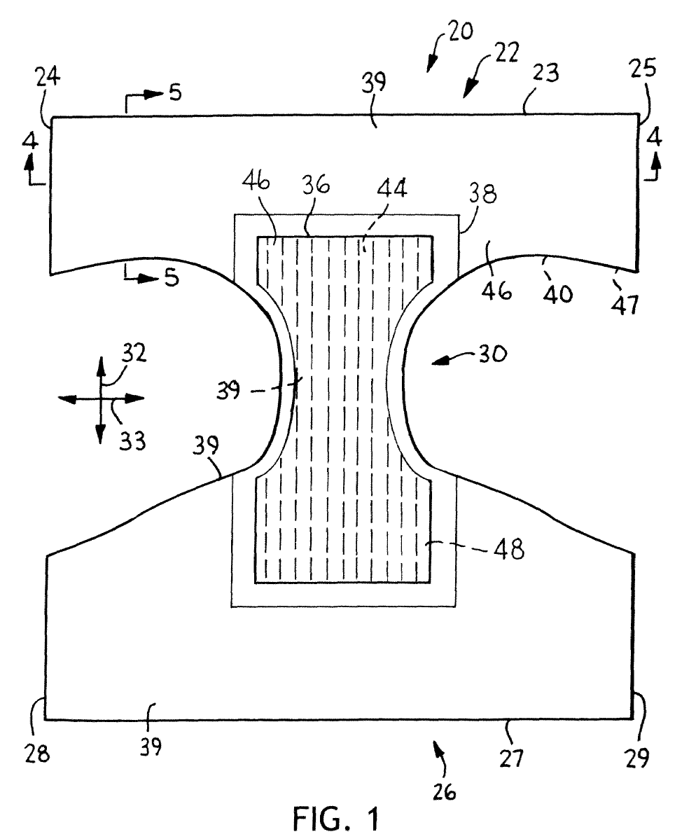

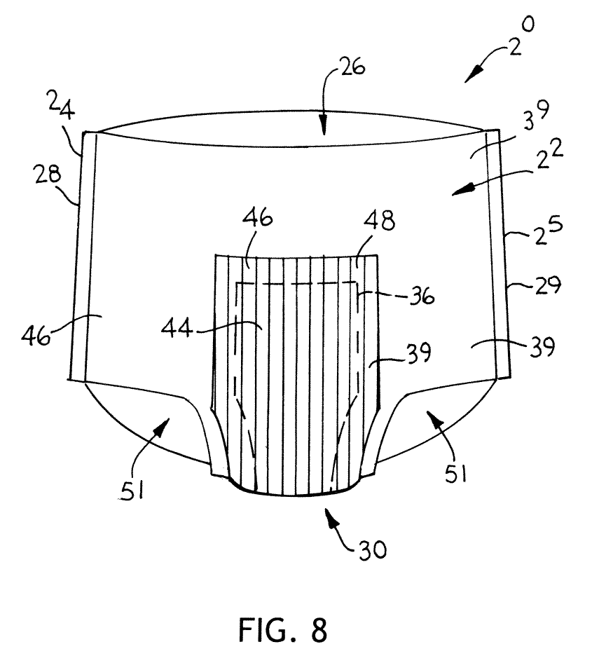

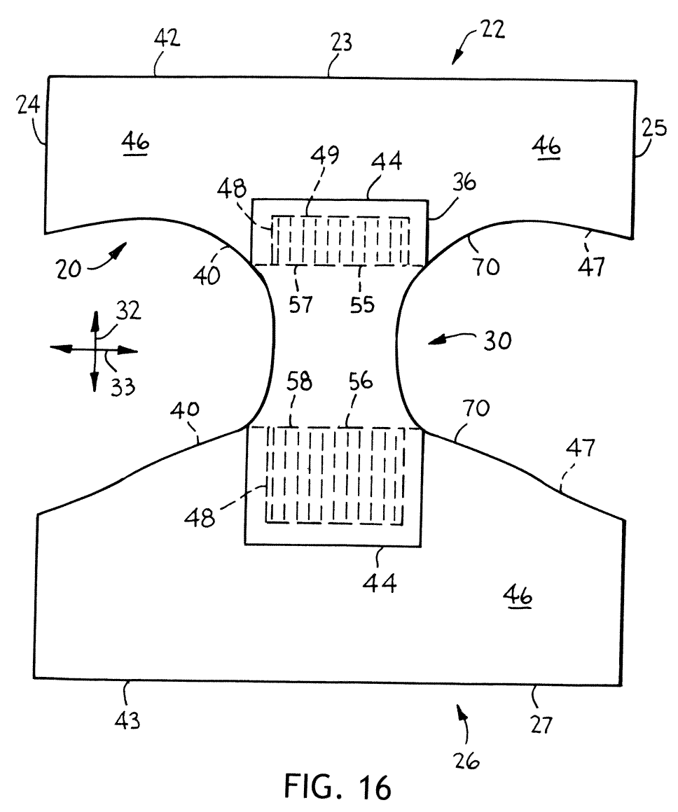

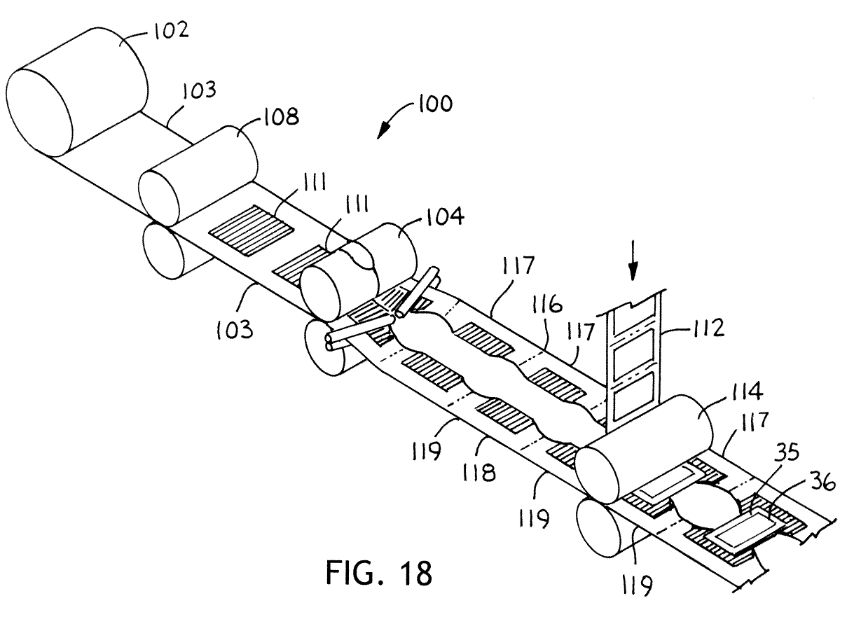

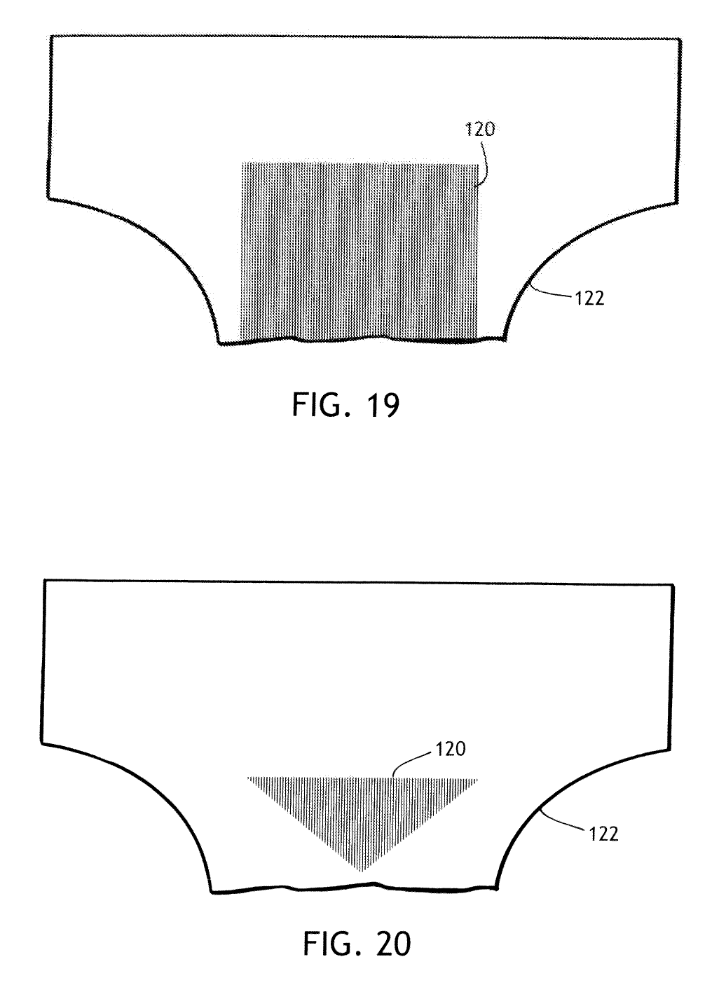

Reference to FIGS. 1 to 25 shall be made in describing various aspects and embodiments of the invention. It should be noted that the embodiments depicted in FIGS. 1-25 are merely representative examples of the garment (or portions of a garment) and process of the invention. Although for illustrative purposes certain features of the present invention shall be described and illustrated with respect to an adult incontinence garment, the various aspects and embodiments of the present invention are also suitable for use with diapers, swim pants, training pants, enuresis garments, and the like.

Referring to FIG. 1, a particular embodiment of an absorbent garment 20 of the present invention defines a front region 22 having a front edge 23 and transversely opposed first and second front side edges 24 and 25, a back region 26 having a back edge 27 and transversely opposed first and second back side edges 28 and 29, and a crotch region 30 disposed longitudinally between and which interconnects the front and back regions 22 and 26. The absorbent garment defines a longitudinal direction 32 which extends from the front region 22 to the back region 26, and a transverse direction 33 which is perpendicular to the longitudinal direction 32. It should be noted that the use of the terms “front” and “back,” such as with “front region” and “back region” and “front edge” and “back edge” is merely for reference purposes in describing and claiming the garment and method of the present invention, and does not imply that the region or edge align with any particular position on the wearer. Thus, for example, the “front region” of a garment as described and claimed herein could be that region of the product that is intended to be positioned on the wearer's front waist, or on the wearer's back waist. Likewise, the “back region” of the garment could be that region of the product that is intended to be positioned on the wearer's front waist, or on the wearer's back waist.

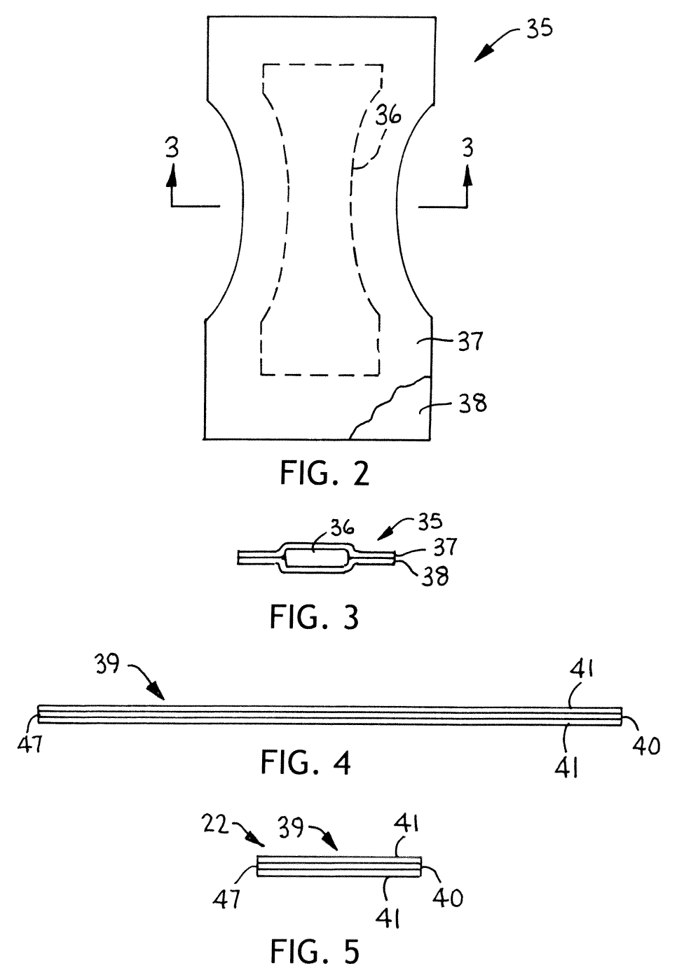

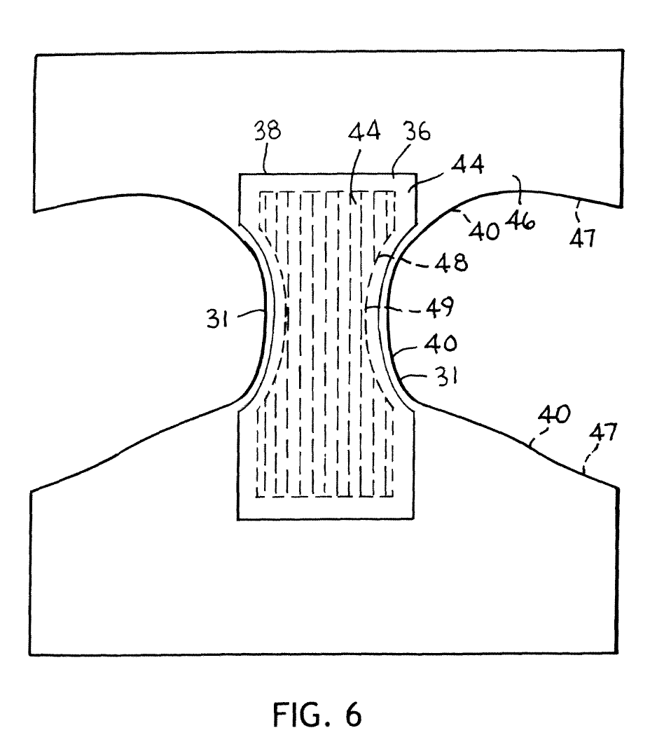

The garment includes an absorbent insert 35 which extends longitudinally from the front region 22 to the back region 26. The absorbent insert 35 includes an absorbent member 36. The absorbent member can be constructed of materials known in the art as suitable for absorbing liquid excretions, such as wood pulp fluff, superabsorbent polymers, absorbent foam, and the like. The absorbent member is, in particular embodiments, encased in one or more substrates. For example, the absorbent member could be wrapped in a tissue and/or a nonwoven substrate. Alternatively, or in addition, the absorbent member can be sandwiched between a garment-side, liquid impermeable backsheet and a body-side, liquid permeable liner. For example, the absorbent insert 35 can be constructed of an absorbent member 36 sandwiched between a liner 37 and a backsheet 38, as representatively illustrated in FIGS. 2 and 3.

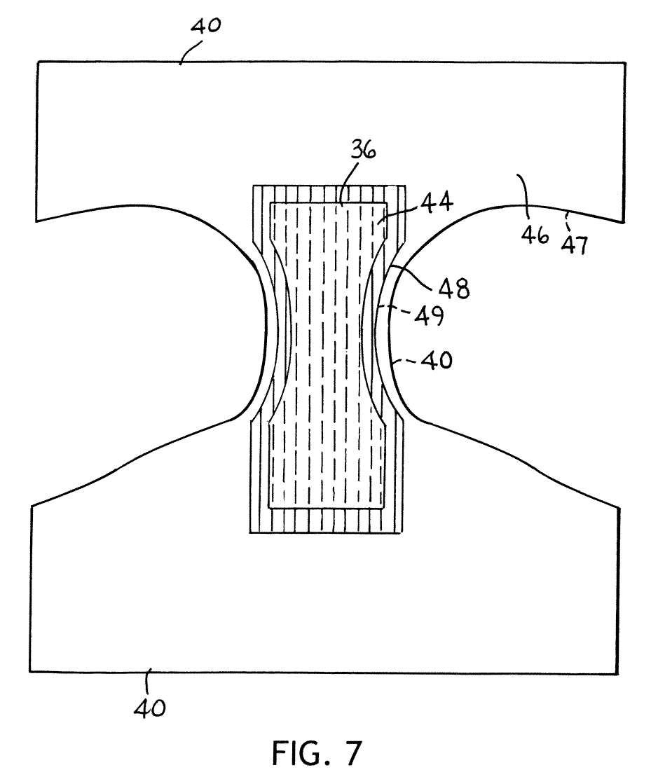

In particular embodiments, the front region 22 is constructed at least in part of a laminate 39 that comprises a polymeric film layer 40 and at least one nonwoven layer 41, wherein both the polymeric film layer 40 and the nonwoven layer 41 extend substantially throughout the entire area of the laminate 39. In one embodiment, representatively illustrated in FIGS. 1, 4, and 5, the laminate 39 comprises two nonwoven layers 41/41 superposed on opposing top and bottom surfaces of the polymeric film 40 such that the polymeric film 40 is sandwiched between the two nonwoven facings 41/41, and both the polymeric film 40 and both nonwoven layers 41/41 extend substantially through the entire area of the laminate 39. In particular embodiments, as representatively illustrated in FIGS. 1 and 4, the laminate 39 transversely extends from the first front side edge 24 to the second front side edge 25.

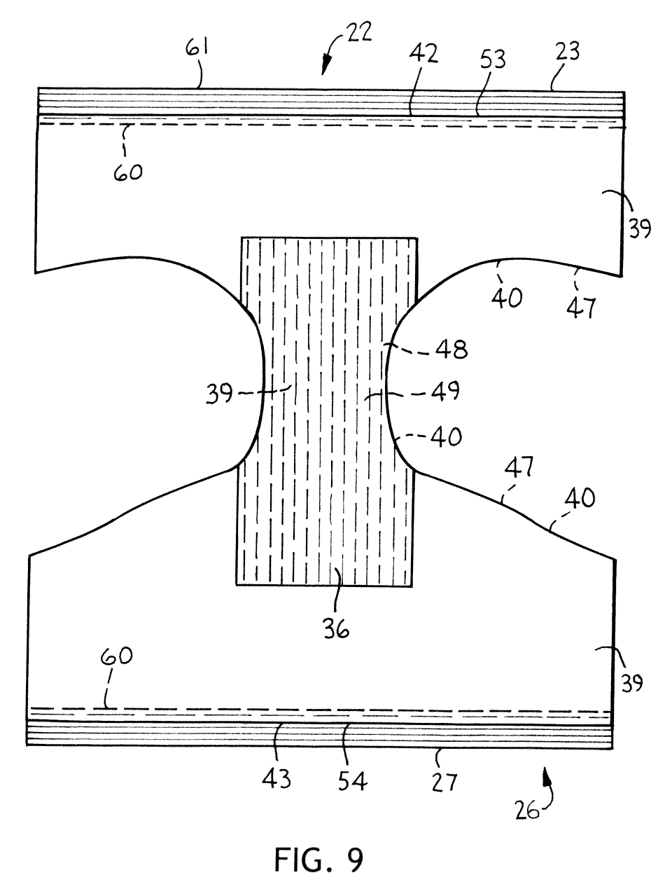

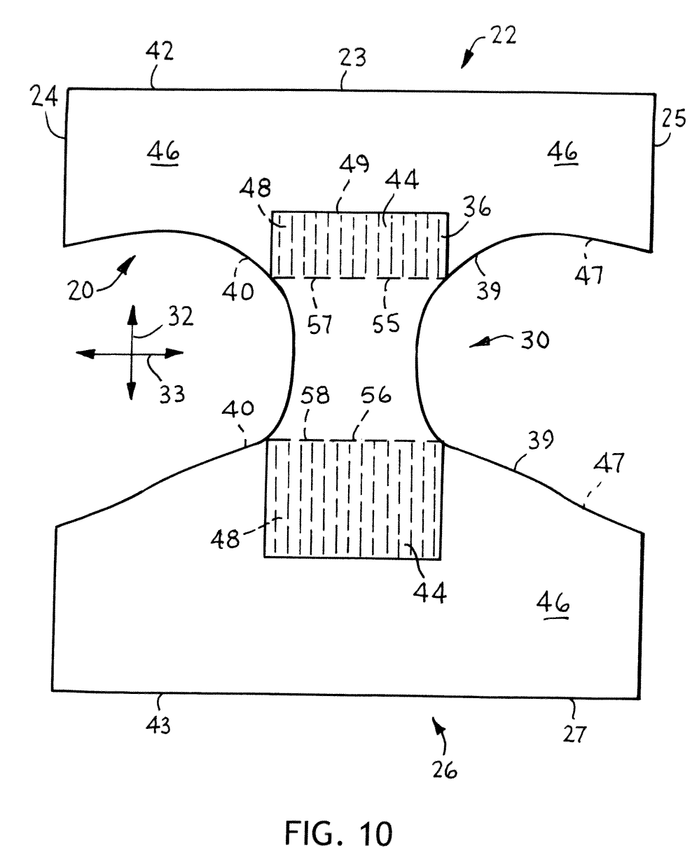

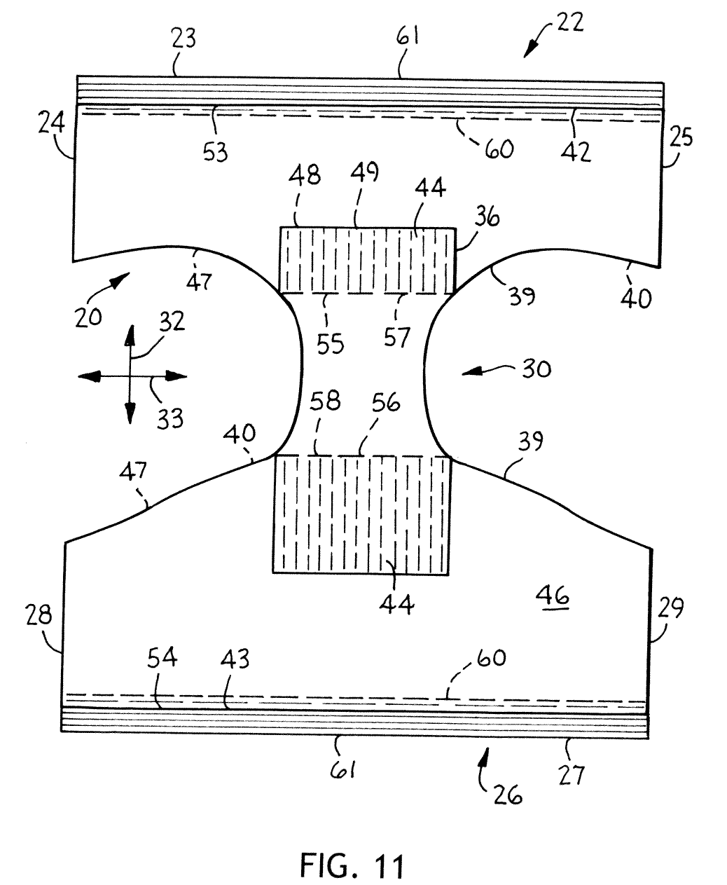

At least a portion of the absorbent member 36 longitudinally overlaps at least a portion of the laminate 39 to define in the laminate 39 an overlapped region 44 and a non-overlapped region 46. At least a portion of the overlapped region 44 of the laminate 39 is non-elastomeric, and at least a portion of the non-overlapped region 46 of the laminate 39 is elastomeric. In FIGS. 6-16, non-elastomeric or partially elastomeric regions are indicated by a pattern of vertically extending lines, which lines are continuous if the non-elastomeric or partially elastomeric regions are exposed, and which lines are dashed if the non-elastomeric or partially elastomeric regions are concealed (hidden) by an overlying component, such as an absorbent member 36. (For simplicity of illustration, only the absorbent member component 36 and the optional backsheet layer 38 of the absorbent insert 35 is depicted in FIGS. 1 and 6; other optional layers, such as a liner 37, is not depicted in FIGS. 1 and 6. Furthermore, in FIGS. 7 and 8, only the absorbent member component 36 of the absorbent insert 35 is depicted.) In one preferred approach, as shall be described in more detail below in conjunction with the method of manufacturing aspect of the present invention, the entire laminate 39 is constructed of an elastomeric film laminate which includes an elastomeric film layer and at least one nonwoven facing layer, and a portion of the laminate has been “deactivated” or “deadened” to render it non-elastomeric. As used herein, “elastomeric” refers to a material or composite which can be elongated by at least 50 percent of its relaxed length and which will recover, upon release of the applied force, at least 50 percent of its elongation. It is generally preferred that the elastomeric material or composite be capable of being elongated by at least 100 percent, more preferably by at least 200 percent, of its relaxed length and recover, upon release of an applied force, at least 50 percent of its elongation. “Non-elastomeric” refers to a material or composite that is non-extensible, or that is extensible but will recover no more than 20 percent of its elongated length after release of an applied elongating force. “Non-extensible” refers to a material that cannot stretch or extend by more than 25 percent of its relaxed length without fracture upon application of a biasing force. “Partially elastomeric” refers to a material or composite which can be elongated by at least 50 percent of its relaxed length and which will recover, upon release of the applied force, more than 20 percent but less than 50 percent of its elongation.

It should be noted that in analyzing an absorbent garment's laminate or regions thereof for extensible or elastic character, the laminate is to be examined removed from separately attached components. For example, before examining the extensible or elastic properties of the overlapped region of the laminate, the absorbent insert, if one is present in the garment, should be removed. For example, an absorbent insert which comprises an absorbent member sandwiched between a liner and backsheet (a common configuration) should be peeled away from the laminate of the front region prior to examining the various regions of the laminate of the front region.

In particular embodiments, the majority of the overlapped region 44 of the laminate 39 is non-elastomeric; that is, more than 50% of the area of the overlapped region 44 of the laminate 39 is non-elastomeric. In preferable embodiments, more than 75%, and still more preferably more than 90% of the area of the overlapped region 44 of the laminate 39 is non-elastomeric. In particular embodiments, the entirety of the overlapped region 44 of the laminate 39 is non-elastomeric. For example, in the embodiment representatively illustrated in FIG. 1, the entirely of the overlapped region 44 of the laminate 39 is non-elastomeric, as the non-elastomeric region 48 is the same size and shape as the absorbent member 36 which overlaps it. In another example, as representatively illustrated in FIG. 6, less than the entirely (but more than 50%) of the overlapped region 44 of the laminate 39 is non-elastomeric, as the absorbent member 36 is larger than the non-elastomeric region 48. For example, in particular embodiments, between 50% and 90%, more preferable between 60% and 90%, and still more preferably between 70% and 90% of the overlapped region 44 of the laminate 39 is non-elastomeric (or, alternatively partially elastomeric). By limiting the size of the non-elastomeric region 48 (such as a “deactivated” region, as more fully explained below) relative to the size of the absorbent member 36 in this manner, it can be possible, in particular embodiments, to desirably increase the total amount of elastomeric stretch material to enhance the fit range of the garment and to induce a bucket-like effect to the absorbent.

In yet another example as representatively illustrated in FIG. 7, the entirety of the overlapped region 44 of the laminate 39 is non-elastomeric, as the non-elastomeric region 48 is actually larger than the absorbent member 36. For example, in particular embodiments, the non-elastomeric region 48 is at least 10% larger, more preferably at least 20% larger, and still more preferably at least 25% larger in area than the overlapped region 44. By employing a non-elastomeric region 48 (such as a “deactivated” region, as more fully explained below) that is larger in area than the amount of overlap in this manner, it can be possible, in particular embodiments, to accommodate the process registration variability that is common to high-speed absorbent article manufacturing techniques. In other words, the footprint of the non-elastomeric region being larger relative to the footprint of the absorbent member (or absorbent member portion in configurations in which the front and back regions are comprised of separate panels) allows for some variability or error in registration as the absorbent insert is mated to the laminate 39. Furthermore, in such embodiments, the “buffer zone” created by the relatively larger non-elastomeric region 48 (larger relative to the overlapped region 44) can assist in minimizing the amount of retraction/bunching directly around the periphery or partial periphery of the absorbent member 36, which can, in certain desirable embodiments, provide a smoother, “flatter,” more underwear-like garment. In particular embodiments, the non-elastomeric region 48 (again, such as a “deactivated” region, as more fully explained below) extends transversely outward from the overlapped region 44 by at least 0.5 inches (1.27 cm), more particularly by at least 0.75 inches (1.9 cm), and still more particularly by about 1 inch (2.54 cm). In particular embodiments, the non-elastomeric region 48 extends longitudinally outward from the overlapped region 44 by at least 0.5 inches (1.27 cm), more particularly by at least 0.75 inches (1.9 cm), and still more particularly by about 1 inch (2.54 cm). In still other embodiments, the non-elastomeric region 48 extends both transversely outward and longitudinally outward from the overlapped region 44 by at least 0.5 inches (1.27 cm), more particularly by at least 0.75 inches (1.9 cm), and still more particularly by about 1 inch (2.54 cm).

In particular embodiments, the majority of the overlapped region 44 of the laminate 39 is only partially-elastomeric; that is, more than 50% of the area of the overlapped region 44 of the laminate 39 is only partially elastomeric. In preferable embodiments, more than 75%, and still more preferably more than 90% of the area of the overlapped region 44 of the laminate 39 is only partially elastomeric. In particular embodiments, the entirety of the overlapped region 44 of the laminate 39 is only partially-elastomeric.

In particular embodiments, substantially the entirety of the area of the overlapped region has an elasticity of less than half, more preferably of less than a quarter, and still more preferably of less than a tenth of the elasticity of substantially the entirety of the area of the non-overlapped portion.

In particular embodiments, the majority of the non-overlapped region 46 of the laminate is elastomeric; that is, more than 50% of the area of the non-overlapped region of the laminate is elastomeric. In preferable embodiments, more than 75%, and still more preferably more than 90% of the area of the non-overlapped region of the laminate is elastomeric. In particular embodiments, the entirety of the non-overlapped region of the laminate is elastomeric. For example, as representatively illustrated in FIG. 1, the entirety of the non-overlapped region 46 of the laminate 39 is elastomeric. In another example, as representatively illustrated in FIG. 7, less than the entirely (but well above 50%) of the non-overlapped region 46 of the laminate 39 is elastomeric.

In particular embodiments, substantially the entirety of the area of the non-overlapped region 46 has an elasticity of more than twice, more preferably of more than three times, and still more preferably more than four times the elasticity of substantially the entirety of the area of the overlapped portion 44. In particular embodiments, elastomeric regions of the laminate are elastomeric in both the transverse direction and the longitudinal direction. In other embodiments, elastomeric regions of the laminate are elastomeric only in the transverse direction.

In particular embodiments, and as representatively illustrated in FIG. 8, the first front side edge 24 is connected to the first back side edge 28, the second front side edge 25 is connected to the second back side edge 29, such that the garment defines a pull-on, pant-like configuration having a waist opening 50 and two leg openings 51. In such an embodiment, the laminate 39 can extend from the front edge to each leg opening, as representatively illustrated in FIG. 8.

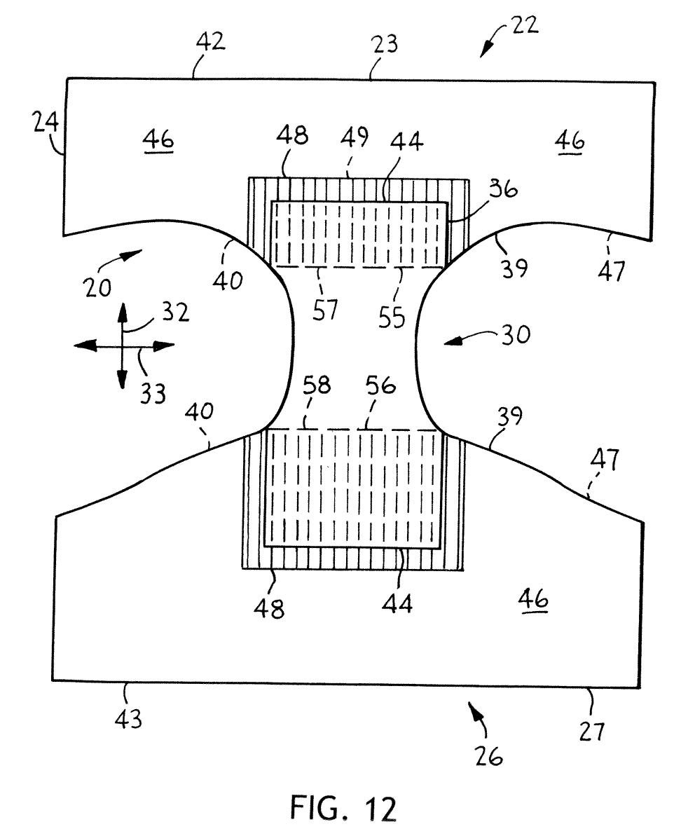

Referring to FIG. 9, the laminate defines a front laminate edge 53 which extends generally parallel to the front edge 23 of the front region 22, and a back laminate edge 54 which extends generally parallel to the back edge 27 of the back region 26. In particular embodiments, an elastomeric waistband 60 is attached to the front laminate edge 53. The longitudinally outermost edge 61 of the elastomeric waistband 60 can be coterminous with the front laminate edge 53 (not shown). Alternatively, as representatively illustrated in FIG. 9, the elastomeric waistband 60 can extend longitudinally between the front laminate edge 53 and the front edge 23, such that the waistband 60 is “cantilevered” off the front laminate edge 53. Similar waistbands could be attached to the back laminate edge 54, or waistbands could be attached to both the front and back laminate edges 53 and 54. Such waistbands are preferably but not necessarily elastomeric, and can comprise elastomeric films, elastomeric strands or ribbons, elastomeric foams, or the like.

In particular embodiments, the back region 26, too, is constructed at least in part of a laminate 39 that comprises a polymeric film layer 40 and a nonwoven layer 41, wherein both the polymeric film layer 40 and the nonwoven layer 41 extend substantially throughout the entire area of the laminate 39. The back region 26 can include any of the features or configurations described above with respect to the front region 22 (for example, with respect to the composition of the laminate, or regarding the elasticity or non-elasticity of the overlapped or non-overlapped regions). Furthermore, both the front and back regions of the garment can simultaneously feature any of the features or configurations described above with respect to the front region 22, alone or in combination. In particular embodiments, the laminate 39 transversely extends from the first front side edge 24 to the second front side edge 25.

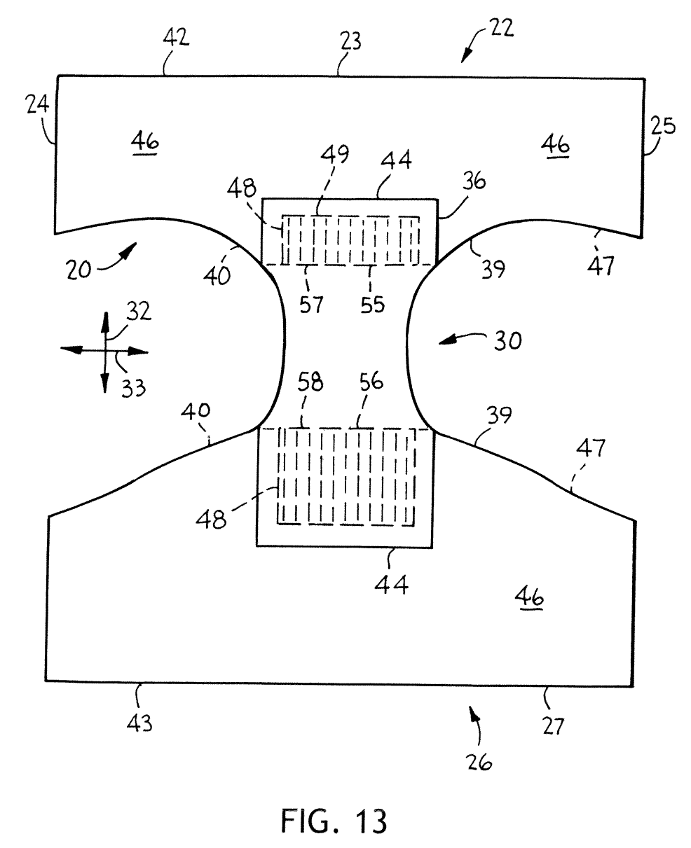

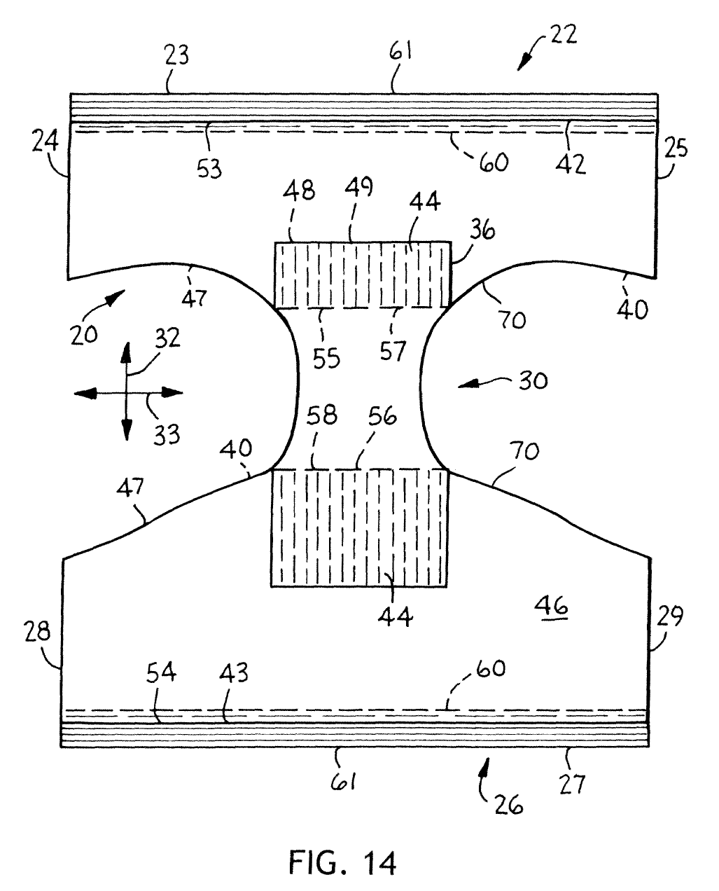

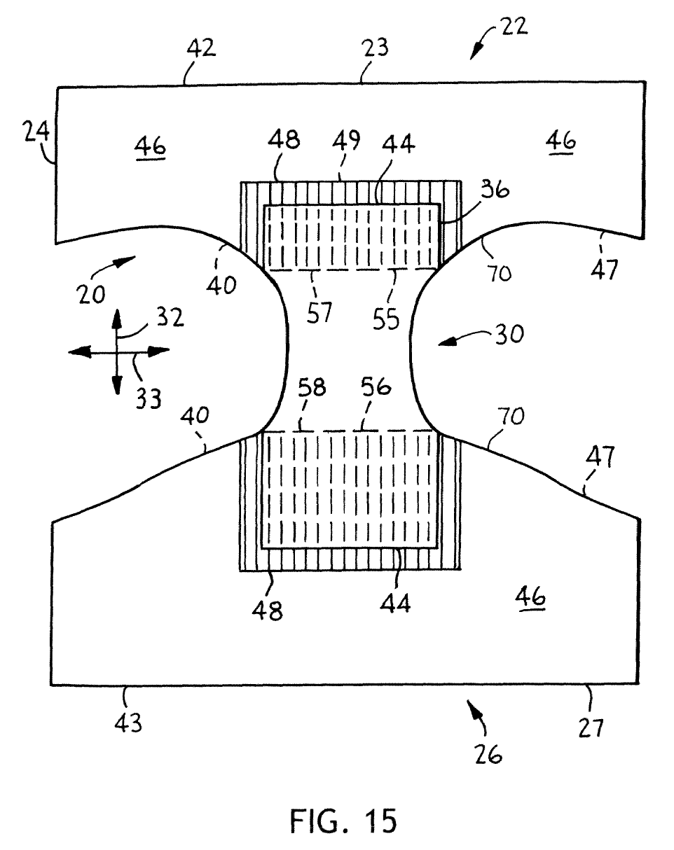

In particular embodiments in which both the front region 22 and the back region 26 comprise a laminate, the laminate 39 can but need not extend from the front region 22 to the back region 26, such that the laminate 39 is continuous and integral through the front region 22, crotch region 30, and back region 26, as representatively illustrated in FIGS. 1 and 7-9. Alternatively, as representatively illustrated in FIGS. 10 and 11, the front region 22 further defines a front crotch edge 55 and the back region 26 further defines a back crotch edge 56, and the front region 22 and the back region 26 are indirectly connected to one another by way of the absorbent member 36. (Note that in FIGS. 10 and 11, as well as in FIGS. 12-16, for simplicity of illustration, only the absorbent member component 36 of the absorbent insert 35 is depicted; other optional layers, such as a backsheet or liner, are not depicted. Furthermore, for simplicity of illustration in FIGS. 10-16, the absorbent member 36 is depicted as extending to and abutting the crotch edges; in commercial practice, the absorbent member 36 need not extend all the way to and abut the crotch edges.)

As noted earlier, the front region 22 preferably comprises a polymeric film layer 40 that transversely and integrally extends from the first front side edge 24 to the second front side edge 25. A series of embodiments shall now be described which are similar to those described above but which shall focus specifically on the polymeric film layer 40. At least a portion of the absorbent member 36 overlaps at least a portion of the film layer 40 to define in the film layer 40 an overlapped region 44 and a non-overlapped region 46. At least a portion of the overlapped region 44 of the polymeric film layer 40 constitutes a non-elastomeric film 49, and at least a portion of the non-overlapped region 46 of the polymeric film layer 40 constitutes an elastomeric film 47.

The present inventors note that polymeric films which are present in laminates are often tightly adhered to the facing layers which frequently form a part of the overall laminate. In such cases, in which, for example, one or more outer nonwoven facings is tightly adhered to and not easily removed from the polymeric film, in analyzing the polymeric film or regions thereof for extensible or elastic character, the polymeric film should be examined with those facing layers still intact and not removed. Nevertheless, as explained above, any components which are separately attached to the laminate should be removed before examining such a laminate. For example, before examining the extensible or elastic properties of the overlapped region of the polymeric film, the absorbent insert (if one is present in the garment) should be removed from the laminate of which the polymeric film is a part as described earlier.

In particular embodiments, the majority of the overlapped region 44 of the polymeric film layer 40 constitutes a non-elastomeric film; that is, more than 50% of the area of the overlapped region 44 of the polymeric film layer 40 constitutes a non-elastomeric film 49. In preferable embodiments, more than 75%, and still more preferably more than 90% of the area of the overlapped region 44 of the polymeric film layer 40 constitutes a non-elastomeric film 49. In particular embodiments, the entirety of the overlapped region 44 of the polymeric film layer 40 constitutes a non-elastomeric film 49.

In particular embodiments, substantially the entirety of the area of the overlapped region 44 of the polymeric film layer 40 has an elasticity of less than half, more preferably of less than a quarter, and still more preferably of less than 10% of the elasticity of substantially the entirety of the area of the non-overlapped region 46 of the polymeric film layer 40.

In particular embodiments, the majority of the non-overlapped region 46 of the polymeric film layer 40 constitutes an elastomeric film 47; that is, more than 50% of the area of the non-overlapped region 46 of the polymeric film layer 40 is elastomeric. In preferable embodiments, more than 75%, and still more preferably more than 90% of the area of the non-overlapped region 46 of the polymeric film layer 40 constitutes an elastomeric film 47. In particular embodiments, the entirety of the non-overlapped region 46 of the polymeric film layer 40 constitutes an elastomeric film 47.

In particular embodiments, substantially the entirety of the area of the non-overlapped region 46 of the polymeric film layer 40 has an elasticity of more than twice, more preferably of more than three times, and still more preferably of more than four times the elasticity of substantially the entirety of the area of the overlapped region 44 of the polymeric film layer 40. In particular embodiments, the elastomeric regions of the polymeric film layer 40 are elastomeric in both the transverse direction and the longitudinal direction. In other embodiments, the elastomeric regions of the polymeric film layer 40 are elastomeric only in the transverse direction. Desirably, the polymeric film layer 40 is sandwiched between two nonwoven facing layers 41 in superposed relation, each nonwoven facing layer 41 being coextensive with the polymeric film layer 40. In certain embodiments, one or more of the nonwoven facing layers 41 can extend beyond the periphery of the polymeric film layer 40, such as to form an integral part of a waistband which extends longitudinally outward from the edge of the polymeric film layer.

In particular embodiments, the first front side edge 24 is connected to the first back side edge 28, and the second front side edge 25 is connected to the second back side edge 29, such that the garment 20 defines a pull-on, pant-like configuration having a waist opening 50 and two leg openings 51. In such an embodiment, the polymeric film layer 40 can extend from the front edge 23 to each leg opening 51.

The polymeric film layer 40 defines a front polymeric film edge 42 which extends generally parallel to the front edge 23 of the front region 22, and a back polymeric film edge 43 which extends generally parallel to the back edge 27 of the back region 26. In particular embodiments, such as those representatively illustrated in FIGS. 9 and 11, an elastomeric waistband 60 is attached to the front polymeric film edge 42. The longitudinally outermost edge 61 of the elastomeric waistband 60 can be coterminous with the front polymeric film edge 42 (not shown). Alternatively, as depicted in FIGS. 9 and 11, the elastomeric waistband 60 can extend longitudinally between the front polymeric film edge 42 and the front edge 23, such that the waistband is “cantilevered” off the front polymeric film edge 42. Similar waistbands could be attached to the back polymeric film edge 43, or waistbands could be attached to both the front and back polymeric film edges. Such waistbands are preferably but not necessarily elastomeric, and can comprise elastomeric films, elastomeric strands or ribbons, elastomeric foams, or the like.

In particular embodiments, the back region 26, too, can comprise a polymeric film layer 40 that transversely and integrally extends from the first back side edge 28 to the second back side edge 29. The back region can feature any of the features described above (for example, with respect elasticity or non-elasticity of the overlapped or non-overlapped regions of the polymeric film layer). Furthermore, both the polymeric film layer 40 in the front and back regions of the garment can simultaneously feature any of the features described above with the respect to the polymeric film layer in the front region, alone or in combination.

In particular embodiments in which both the front region 22 and the back region 26 comprise a polymeric film layer 40 having elastomeric regions, the polymeric film layer 40 can but need not extend from the front region 22 to the back region 26, such that the polymeric film layer 40 is continuous and integral through the front region 22, crotch region 30, and back region 26, as is representatively illustrated in FIGS. 1, 6, and 9. Alternatively, in particular embodiments, the polymeric film layer in the front region 22 further defines a film front crotch edge 57 and the polymeric film layer in the back region 26 further defines a film back crotch edge 58, and the polymeric film layer in the front region 22 and the polymeric film layer in the back region 26 are indirectly connected to one another by way of the absorbent member 36, as is representatively illustrated in FIGS. 10-13.

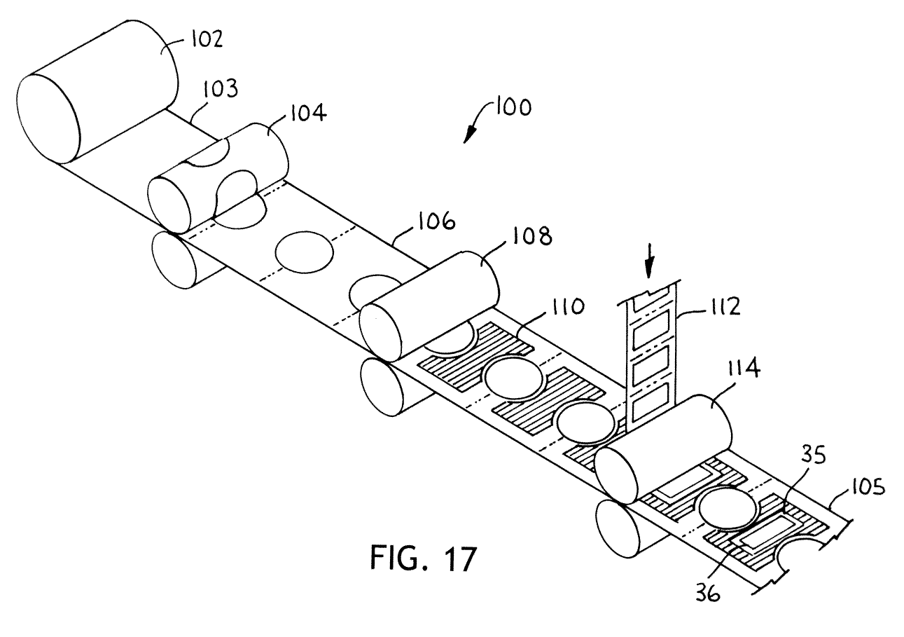

In an alternative way of characterizing certain embodiments of the invention and shall be representatively illustrated in FIGS. 14-16, the front region 22 is constructed of an elastomeric film laminate 70 that comprises an elastomeric film layer and a nonwoven layer, wherein both the elastomeric film layer and the nonwoven layer extend substantially throughout the entire area of the laminate. In one embodiment, the laminate comprises two nonwoven layers superposed on opposing top and bottom surfaces of the elastomeric film layer such that the elastomeric film layer is sandwiched between the two nonwoven facings, and both the elastomeric film layer and both nonwoven layers extend substantially through the entire area of the elastomeric film laminate 70. In particular embodiments, the elastomeric film laminate 70 transversely extends from the first front side edge 24 to the second front side edge 25.

At least a portion of the absorbent member 36 longitudinally overlaps at least a portion of the elastomeric film laminate 70 to define in the laminate 70 an overlapped region 44 and a non-overlapped region 46. At least a portion 74 of the overlapped region 44 of the elastomeric film laminate 70 is deactivated, and at least a portion 76 of the non-overlapped region 46 of the elastomeric film laminate 70 is elastomeric. “Deactivated” as used herein to describe a material, region of a material, or regions of a material means that the material, region, or regions of material has been treated in some way (examples of which shall be described below) to substantially destroy the elastic properties of the material, region, or regions, rendering the material, region, or regions non-elastomeric.

It should be noted that in analyzing the elastomeric film laminate or regions thereof for deactivation or for elastic character, the laminate is to be examined removed from separately attached components. For example, before examining the deactivation or elastic properties of the overlapped region of the laminate, the absorbent insert, if one is present in the garment, should be removed. For example, an absorbent insert which comprises an absorbent member sandwiched between a liner and backsheet (a common configuration) should be peeled away from the laminate of the front region prior to examining the various regions of the front region.

In particular embodiments, the majority of the overlapped region 44 of the elastomeric film laminate 70 is deactivated; that is, more than 50% of the area of the overlapped region 44 of the elastomeric film laminate 70 is deactivated. In preferable embodiments, more than 75%, and still more preferably more than 90% of the area of the overlapped region 44 of the elastomeric film laminate 70 is deactivated. In particular embodiments, the entirety of the overlapped region 44 of the elastomeric film laminate 70 is deactivated.

In particular embodiments, the majority of the non-overlapped region 46 of the elastomeric film laminate 70 is elastomeric; that is, more than 50% of the area of the non-overlapped region 46 of the elastomeric film laminate 70 is elastomeric. In preferable embodiments, more than 75%, and still more preferably more than 90% of the area of the non-overlapped region 46 of the elastomeric film laminate 70 is elastomeric. In particular embodiments, the entirety of the non-overlapped region 46 of the elastomeric film laminate 70 is elastomeric. In certain embodiments, elastomeric regions of the elastomeric film laminate 70 are elastomeric in both the transverse direction and the longitudinal direction.

In particular embodiments, the first front side edge 24 is connected to the first back side edge 28, and the second front side edge 25 is connected to the second back side edge 29, such that the garment 20 is in a pull-on, pant-like configuration having a waist opening 50 and two leg openings 51. In such an embodiment, the elastomeric film laminate 70 can extend from the front edge 23 to each leg opening 51.

The elastomeric film laminate 70 defines a front laminate edge 53 which extends generally parallel to the front edge of the front region. An additional elastomeric film laminate 70 in the back waist region, if any, can define a back laminate edge 54 which extends generally parallel to the back edge of the back region. In particular embodiments, an elastomeric waistband 60 is attached to the front laminate edge 53. The longitudinally outermost edge 61 of the elastomeric waistband 60 can be coterminous with the front laminate edge 53. Alternatively, the elastomeric waistband 60 can extend longitudinally between the front laminate edge 53 and the front edge 23, such that the waistband is “cantilevered” off the front laminate edge 53. Similar waistbands could be attached to the back laminate edge 54, or waistbands could be attached to both the front and back laminate edges. Such waistbands are preferably but not necessarily elastomeric, and can comprise elastomeric films, elastomeric strands or ribbons, elastomeric foams, or the like.

In particular embodiments, the back region, too, is constructed at least in part of an elastomeric film laminate that comprises a polymeric film layer and a nonwoven layer, wherein both the polymeric film layer and the nonwoven layer extend substantially throughout the entire area of the elastomeric film laminate. The back region can feature any of the features described above (for example, with respect to the configuration of the elastomeric film laminate, or regarding the elastomeric or deactivation of the overlapped or non-overlapped regions). Furthermore, both the front and back regions of the garment can simultaneously feature any of the features described above with respect to the front region, alone or in combination.

In particular embodiments in which both the front region and the back region comprise an elastomeric film laminate, the elastomeric film laminate 70 can but need not extend from the front region 22 to the back region 26, such that the elastomeric film laminate 70 is continuous and integral through the front region 22, crotch region 30, and back region 26. In such an embodiment, the region of the elastomeric film laminate 70 within the crotch region that is overlapped by the absorbent member 36 can but need not be deactivated. Further, in such an embodiment, the region of the laminate within the crotch region that is deactivated desirably does not transversely extend to the transverse side edges 31, as representatively illustrated in FIG. 6. Such a configuration will allow the outer edges of the elastomeric film laminate 70 within the crotch region 30 to retract inward, thus placing a force on the garment that tends to urge it upward toward the body and forming a “bucket” to better help contain fluid. Alternatively, in particular embodiments employing an elastomeric film laminate 70 having one or more deactivated portions 74, the front region 22 further defines a front crotch edge 55 and the back region 26 further defines a back crotch edge 56, and the front region 22 and the back region 26 are indirectly connected to one another by way of the absorbent member 36, as representatively illustrated in FIGS. 14-16.

A process 100 for making an absorbent garment of the present invention, such as those representatively described above, shall now be explained. One embodiment of the process includes providing an hourglass shaped panel comprising an elastomeric film laminate. Examples of suitable elastomeric film laminates include those described above in conjunction with the discussion of the garment aspect of the present invention. In particular embodiments, the laminate comprises two nonwoven layers superposed on opposing top and bottom surfaces of an elastomeric polymeric film such that the polymeric film is sandwiched between the two nonwoven facings, wherein both the polymeric film and both nonwoven layers extend substantially through the entire area of each laminate. The panel defines a front waist edge, a back waist edge, and first and second side edges extending between and interconnecting the front waist edge and the back waist edge. The elastomeric film laminate extends from the front waist edge to the back waist edge and extends from the first side edge to the second side edge.

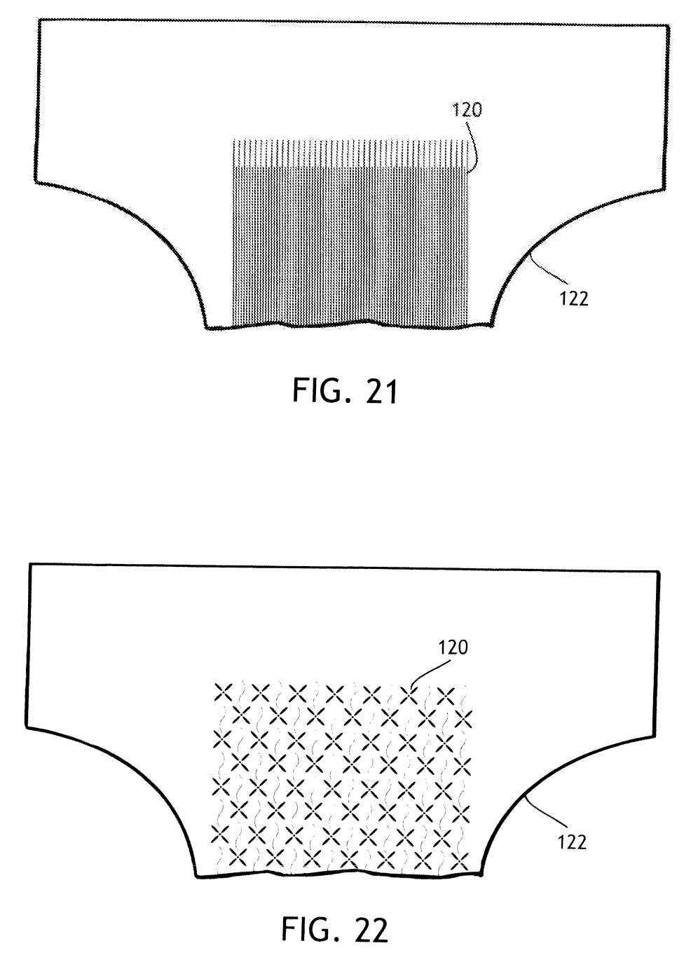

One technique, representatively illustrated in FIG. 17, to generate the hourglass shaped panel is to begin with a roll supply 102 of an elastomeric film laminate web 103. After the laminate is unwound, trim portions are removed by a cutter 104 to define an interconnected series of hourglass shaped panels 106. Individual hourglass shaped panels are eventually cut from the interconnected series of panels (not shown).

The process 100 further includes deactivating a central region of each panel in the interconnected series 106 via a deactivation unit 108 to create deactivated regions 110 in the elastomeric film laminate web 103. The deactivation can be accomplished by any of a variety of means. Frequently, some form of energy is applied to deactivate the central region of the each panel in the series, such as pressure, heat, ultrasonic energy, combinations thereof, and the like. Techniques employing pressure, heat, and ultrasonic energy are known in the art. The deactivation can occur in a variety of patterns. For example, the deactivating energy could be applied in a solid pattern, a series of vertical stripes, horizontal stripes, or diagonal stripes, a series of squares or dots, or other suitable pattern. Further detail regarding the use of patterns to impart the desired deactivation is provided below.

The process 100 further includes providing an absorbent insert 35. The insert comprises an absorbent member 36 as described earlier. One example of a suitable insert 35 comprises a bodyside liner, a garment-side backsheet, and an absorbent insert comprised of wood pulp fluff and superabsorbent polymer. In particular embodiments, each of these components is introduced into the process in continuous fashion, resulting in an interconnected series 112 of absorbent inserts 35 that is fed into the rest of the process 100, such as representatively illustrated in FIG. 17. Techniques to assemble an interconnected series 112 of absorbent inserts 35, such by sandwiching an absorbent member 36 between backsheet and liner layers, or by providing a thin, integral absorbent in roll form, such as an absorbent foam, are known in the art and are therefore not shown or discussed in detail herein.

The process 100 further includes attaching each absorbent insert to a panel 105, such that a portion of the absorbent member 36 overlaps at least a portion of the deactivated region 110. For example, the absorbent insert can be attached to the panel at an attachment station 114, at which each absorbent insert is cut from the interconnected series 112 and applied to each panel 105 in the interconnected series of panels 112. In one embodiment, the absorbent member 36 overlaps the entirety of the deactivated region. In certain embodiments, the area of the deactivated region is at least 10% smaller than the area of the absorbent member. In other embodiments, the absorbent member and the deactivated region have substantially the same shape and area. In still other embodiments, such as that representatively illustrated in FIG. 17, the area of the panel 105 that is overlapped by the absorbent member 36 is smaller than the area of the deactivated region 110. For example, in one embodiment, the area of the panel 105 that is overlapped by the absorbent member 36 is no more 80% that of the area of the deactivated region 110. Alternatively, any of the configurations described earlier in conjunction with the discussion of the deactivated regions of the garment aspect of the invention are suitable for use with the presently discussed process.

The process can further include attaching a front elastomeric waistband to the front waist edge, and can further include attaching a back elastomeric waistband to the back waist edge (not shown). Each elastomeric waistband edge can be positioned to be flush with the waist edge to which it is attached. Alternatively, as discussed earlier, each elastomeric waistband can be attached such that the front elastomeric waistband extends longitudinally outward from the front waist edge and such that the back elastomeric waistband extends longitudinally outward from the back waist edge, in a “cantilevered” manner.

Each panel 105 is eventually cut (not shown) from the interconnected series of panels 106 and, in particular embodiments, a first front side edge of each panel 105 is connected to a first back side edge of each panel, and a second front side edge of each panel 105 is connected to a second back side edge of each panel, such that the garment is in a pull-on, pant-like configuration having a waist opening and two leg openings.

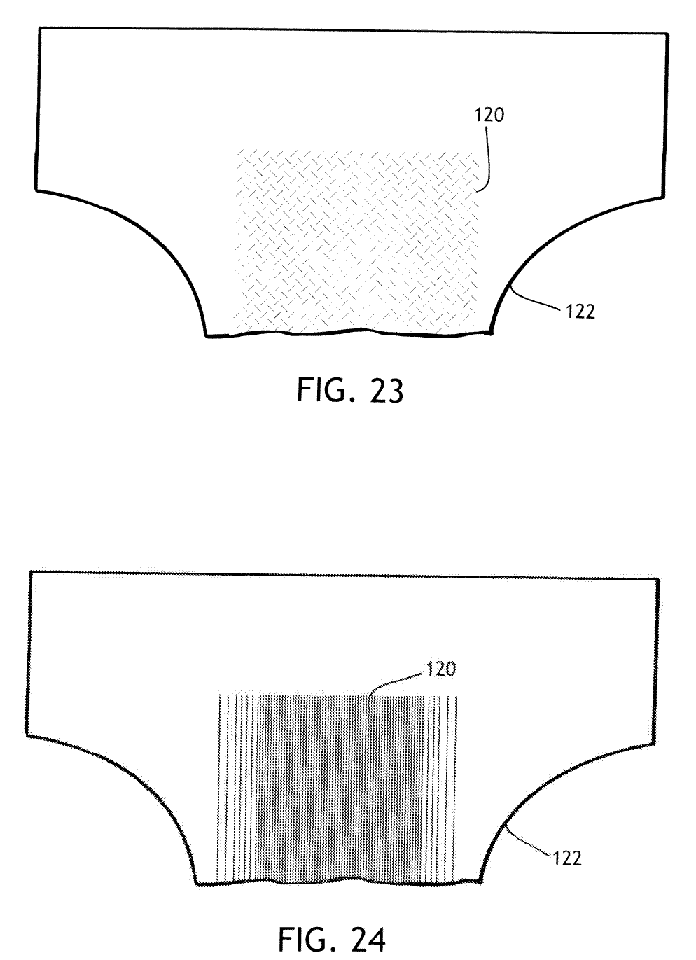

An alternative configuration of the process aspect of the present invention, representatively illustrated in FIG. 18, includes providing a front panel comprising an elastomeric film laminate and defining a waist edge and a crotch edge, and providing a back panel comprising an elastomeric film laminate and defining a waist edge and a crotch edge. (Examples of suitable elastomeric film laminates include those described above in conjunction with the discussion of the garment aspect of the present invention.) For example, a roll 102 of an elastomeric film laminate web 103 is unwound. The process 100 further includes deactivating regions 111 of the laminate web 103 via a deactivation unit 108 to create deactivated regions 111 in the elastomeric film laminate web 103. Any of the deactivation techniques and patterns described above would be suitable to perform the deactivation in this embodiment. Additionally, the laminate web 103 is shaped and slit into two separate webs 116/118 via a cutter 104; namely, the web 103 is split into a front panel web 116 defining an interconnected series of front panels 117 (each having a front panel deactivated region 120) and a back panel web 118 defining an interconnected series of back panels 119 (each having a back panel deactivated region 121). An absorbent insert 35 comprising an absorbent member 36 (such as described earlier) is provided and is attached to each front panel 122 and each back panel 123, such that the absorbent insert 35 extends between the front panel 122 and the back panel 123, and such that a front portion of the absorbent member overlaps at least a portion of the front panel deactivated region 120, and such that a back portion of the absorbent member overlaps at least a portion of the back panel deactivated region 12. In certain embodiments, the front portion of the absorbent member overlaps the entirety of the front panel deactivated region, and the back portion of the absorbent member overlaps the entirety of the back panel deactivated region. In other embodiments, such as that representatively illustrated in FIG. 18, the area of the front panel 122 that is overlapped by the front portion of the absorbent member is smaller than the area of the front panel deactivated region 120, and the area of the back panel 123 that is overlapped by the back portion of the absorbent member is smaller than the area of the back panel deactivated region 121. For example, in one embodiment, the area of each front panel 122 that is overlapped by the front portion of the absorbent member is no more than 80% that of the area of the front panel deactivated region 120, and the area of the back panel 123 that is overlapped by the back portion of the absorbent member is at most 80% that of the area of the back panel deactivated region 121.

In certain embodiments, the front panel web 116 and the back panel web 118 can be provided separately, and one or both of such separately provided webs can be separately subjected to a deactivation step to introduce one or more deactivated regions (not shown). Alternatively, any of the configurations described earlier in conjunction with the discussion of the deactivated regions of the garment aspect of the invention in which the front and back panels are separate and not integral with one another are suitable for use with the presently discussed embodiment of the process aspect of the invention. Furthermore, elastomeric waistbands can be attached the waist edge of the front panel, the back panel, or both, in any of the manner described above (not shown). In particular embodiments, an elastomeric waistband can be formed integrally with the elastomeric film laminate of the front or back panels, such as by longitudinally folding the end edge of a waist panel upon itself, perhaps sandwiching one or more elastomeric strands or ribbons within the fold.

As noted above, the deactivation step of the process can be performed using a variety of deactivation patterns. For example, the deactivating energy could be applied in a solid pattern, a series of vertical stripes, horizontal stripes, or diagonal stripes, a series of squares or dots, or other suitable pattern. One advantage to using a certain deactivation “patterns” (as opposed to “flat,” continuous approach to deactivation) is that at the high speeds at which modern absorbent article processes frequently operate, it has been found to be difficult to transfer enough energy (such as heat or ultrasonic energy) to the moving web to satisfactorily deactivate the region of interest in what is typically fractions of a second. We have found that the use of certain deactivation patterns not only reduces the amount of energy needed to deactivate, but can assist in delivering a satisfactory level of deactivation even at high processing speeds.

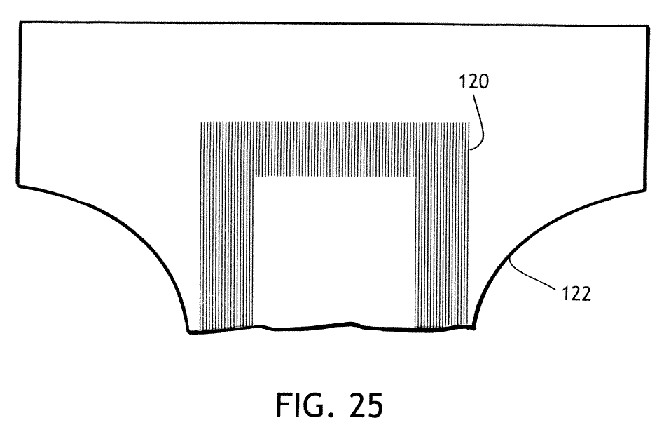

Examples of certain patterns that we believe to offer advantage in this regard are representatively illustrated in FIGS. 19-25. Although each of the patterns is representatively illustrated upon a front region panel of a garment, each of these representative patterns could also be employed on a back region panel. FIG. 19 depicts a pattern comprised of a series of thin, vertical lines or bars. Such a pattern is desirable because it allows thin regions of the elastomeric laminate or elastomeric film being deactivated to retain some amount of their elasticity. Such an approach can deliver much of the benefit of deactivating part or all of the overlapped region as discussed earlier, but can also allow those thin regions of partial retained elasticity to contribute to the overall stretch capability of the garment, delivering increased versatility to the garment in terms of fitting wearers of different sizes. FIG. 20 depicts a pattern similar to the pattern depicted in FIG. 19 but the footprint of the pattern of FIG. 20 is confined to a triangular-spaced area. Such a pattern is desirable because it can assist in urging a bucket-like shape to the absorbent member by selectively introducing elastomeric forces to specific portions of the absorbent member 36. FIG. 22 depicts a pattern similar to the pattern depicted in FIG. 19, but the top portion of the pattern of FIG. 21 employs a line spacing which is less dense than the line spacing in the remainder of the pattern. Such a pattern is desirable because it can provide a region of transition from the more heavily deactivated area to the non-deactivated area of the elastomeric material. While providing some elastomeric character at the edge of the absorbent member 36, this “transition” region of deactivation can prevent too much retraction from occurring at the edge of the absorbent member, and thereby lessen bunching and buckling of the absorbent at the edges. FIG. 24 depicts a pattern that is similar to the pattern depicted FIG. 19, but the side portions of the pattern of FIG. 24 employs a line spacing which is less dense than the line spacing in the remainder of the pattern. Such a pattern is desirable because it can provide a region of transition from the more heavily deactivated area to the non-deactivated area of the elastomeric material. While providing some elastomeric character at the edge of the absorbent member 36, this “transition” region of deactivation can prevent too much retraction from occurring at the edge of the absorbent member, and thereby lessen bunching and buckling of the absorbent at the edges. FIG. 22 depicts a pattern comprised of four-pointed stars and short S-shaped lines. Such a pattern would be aesthetically desirable for certain female-specific embodiments. FIG. 23 depicts a pattern comprised of a series of short line segments positioned at 90 degrees to each other, and all of which lie at an angle of roughly 45 degrees to the longitudinal axis. Such a pattern would be aesthetically desirable for certain male-specific embodiments.

In an alternative embodiment of the present invention, the majority of the overlapped region 44 is not deactivated, but the elastomeric character is instead preserved. However, to reduce the problem of bunching at the edges of the absorbent member 36, the area of the elastomeric film laminate that surrounds the absorbent member 36 is non-elastomeric, such as by being deactivated. For example, a U-shaped deactivation pattern 120 is representatively illustrated in FIG. 25. Such as pattern would surround the end region of the absorbent core, and act as a “buffer zone” or “transition” region to minimize bunching at the edges of the absorbent member 36. Such a U-shaped deactivation pattern could partially overlap with the absorbent member 36. In other embodiments, such a U-shaped deactivation pattern would be substantially non-overlapping with the absorbent member 36.

It will be appreciated that details of the foregoing embodiments, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention, which is defined in the following claims and all equivalents thereto. Further, it is recognized that many embodiments may be conceived that do not achieve all of the advantages of some embodiments, particularly of the preferred embodiments, yet the absence of a particular advantage shall not be construed to necessarily mean that such an embodiment is outside the scope of the present invention.

We claim:

1. An absorbent garment defining a longitudinal direction and a transverse direction, the absorbent garment comprising:
a front region defining a front edge and first and second transversely opposed front side edges;
a back region defining a back edge and first and second transversely opposed back side edges;
a crotch region disposed longitudinally between and interconnecting the front and back regions;
an absorbent member extending longitudinally from the front region to the back region;
wherein the front region comprises a laminate that comprises a polymeric film layer and a nonwoven layer, wherein both the polymeric film layer and the nonwoven layer extend substantially throughout the entire area of the laminate;
wherein at least a portion of the absorbent member longitudinally overlaps at least a portion of the laminate to define in the laminate an overlapped region and a non-overlapped region;
wherein at least a portion of the overlapped region of the laminate is non-elastomeric, and wherein at least a portion of the non-overlapped region of the laminate is elastomeric.

2. The absorbent garment of claim 1, wherein the majority of the overlapped region of the laminate is non-elastomeric.

3. The absorbent garment of claim 2, wherein the entirety of the overlapped region of the laminate is non-elastomeric.

4. The absorbent garment of claim 3, wherein the overlapped region has an elasticity of no more than 80% of the elasticity of elastomeric portions of the non-overlapped region.

5. The absorbent garment of claim 1, wherein the majority of the non-overlapped region of the laminate is elastomeric.

6. The absorbent garment of claim 5, wherein the entirety of the non-overlapped region of the laminate is elastomeric.

7. The absorbent garment of claim 6, wherein the non-overlapped region has an elasticity at least 20% greater than the elasticity of non-elastomeric portions of the overlapped region.

8. The absorbent garment of claim 1, wherein the laminate comprises two nonwoven layers superposed on opposing top and bottom surfaces of the polymeric film such that the polymeric film is sandwiched between the two nonwoven facings, wherein both the polymeric film and both nonwoven layers extend substantially through the entire area of the laminate.

9. The absorbent garment of claim 1, wherein elastomeric regions of the laminate are elastomeric in both the transverse direction and the longitudinal direction.

10. The absorbent garment of claim 1, wherein the first front side edge is connected to the first back side edge and wherein the second front side edge is connected to the second back side edge, such that the garment is in a pull-on, pant-like configuration having a waist opening and two leg openings.

11. The absorbent garment of claim 1, wherein the laminate extends from the front edge to each leg opening.

12. The absorbent garment of claim 1, wherein the laminate defines a front laminate edge which extends generally parallel to the front edge, and wherein an elastomeric waistband is attached to the front laminate edge and extends longitudinally between the front laminate edge and the front edge.

13. The absorbent garment of claim 1, wherein the laminate transversely extends from the first front side edge to the second front side edge.

14. The absorbent garment of claim 1, wherein the back region comprises a laminate that comprises a polymeric film layer and a nonwoven layer, wherein both the polymeric film layer and the nonwoven layer extend substantially throughout the entire area of the laminate of the back region;
wherein at least a portion of the absorbent member longitudinally overlaps at least a portion of the laminate of the back region to define in the laminate of the back region an overlapped region and a non-overlapped region;
wherein at least a portion of the overlapped region of the laminate of the back region is non-elastomeric, and wherein at least a portion of the non-overlapped region of the laminate of the back region is elastomeric.

15. The absorbent garment of claim 14, wherein the laminate extends from the front region to the back region, such that the laminate is continuous and integral through the front region, crotch region, and back region.

16. The absorbent garment of claim 14, wherein the front region further defines a front crotch edge and the back region further defines a back crotch edge, such that the front region and the back region are indirectly connected to one another by way of the absorbent member.

17. An absorbent garment defining a longitudinal direction and a transverse direction, the absorbent garment comprising:
a front region defining a front edge and first and second transversely opposed front side edges;
a back region defining a back edge and first and second transversely opposed back side edges;
a crotch region disposed longitudinally between and interconnecting the front and back regions;
an absorbent member extending longitudinally from the front region to the back region;
wherein the front region comprises a polymeric film transversely extending from the first front side edge to the second front side edge;
wherein at least a portion of the absorbent member overlaps at least a portion of the film to define in the film an overlapped region and a non-overlapped region;
wherein at least a portion of the overlapped region of the polymeric film constitutes a non-elastomeric film, and wherein at least a portion of the non-overlapped region of the polymeric film constitutes an elastomeric film.

18. The absorbent garment of claim 17, wherein the majority of the overlapped region of the polymeric film constitutes a non-elastomeric film.

19. The absorbent garment of claim 17, wherein the entirety of the overlapped region of the polymeric film constitutes a non-elastomeric film.

20. The absorbent garment of claim 19, wherein the overlapped region has an elasticity of no more than 80% of the elasticity of elastomeric portions of the non-overlapped region.

21. The absorbent garment of claim 17, wherein the majority of the non-overlapped region of the polymeric film constitutes an elastomeric film.

22. The absorbent garment of claim 17, wherein substantially the entirety of the non-overlapped region of the polymeric film constitutes an elastomeric film.

23. The absorbent garment of claim 22, wherein the non-overlapped region has an elasticity at least 20% greater than the elasticity of non-elastomeric portions of the overlapped region.

24. The absorbent garment of claim 17, wherein the polymeric film is sandwiched between two nonwoven facings in superposed relation, each nonwoven facing being coextensive with the polymeric film.

25. The absorbent garment of claim 17, wherein the first front side edge is connected to the first back side edge and wherein the second front side edge is connected to the second back side edge, such that the garment is in a pull-on, pant-like configuration having a waist opening and two leg openings.

26. The absorbent garment of claim 17, wherein the elastomeric film defines a front elastomeric film edge, and wherein an elastomeric waistband is attached to the front elastomeric film edge and extends longitudinally between the front elastomeric film edge and the front edge.

27. The absorbent garment of claim 17, wherein the back region comprises a polymeric film transversely extending from the first back side edge to the second back side edge;
wherein at least a portion of the absorbent member overlaps at least a portion of the film in the back region to define in the film in the back region an overlapped region and a non-overlapped region;
wherein at least a portion of the overlapped region of the polymeric film in the back region constitutes a non-elastomeric film, and wherein at least a portion of the non-overlapped region of the polymeric film in the back region constitutes an elastomeric film.

28. The absorbent garment of claim 27, wherein the polymeric film extends from the front region to the back region, such that the polymeric film is continuous and integral through the front region, crotch region, and back region.

29. The absorbent garment of claim 27, wherein the front region further defines a front crotch edge and the back region further defines a back crotch edge, such that the front region and the back region are indirectly connected to one another by way of the absorbent member.

30. An absorbent garment defining a longitudinal direction and a transverse direction, the absorbent garment comprising:
a front region defining a front edge and first and second transversely opposed front side edges;
a back region defining a back edge and first and second transversely opposed back side edges;
a crotch region disposed longitudinally between and interconnecting the front and back regions;
an absorbent member extending longitudinally from the front region to the back region;
wherein the front region comprises an elastomeric film laminate that comprises an elastomeric film layer and a nonwoven layer, wherein both the elastomeric film layer and the nonwoven layer extend substantially throughout the entire area of the laminate;
wherein at least a portion of the absorbent member longitudinally overlaps at least a portion of the laminate to define in the laminate an overlapped region and a non-overlapped region;
wherein at least a portion of the overlapped region of the elastomeric film laminate is deactivated, and wherein at least a portion of the non-overlapped region of the elastomeric film laminate is elastomeric.

31. The absorbent garment of claim 30, wherein the majority of the overlapped region of the laminate is deactivated.

32. The absorbent garment of claim 31, wherein the entirety of the overlapped region of the laminate is deactivated.

33. The absorbent garment of claim 32, wherein the elastomeric film defines a deactivated region, and the deactivated region is larger than the overlapped portion.

34. The absorbent garment of claim 33, wherein the deactivated region is at least 20% larger than the overlapped portion.

35. The absorbent garment of claim 33, wherein the deactivated region extends transversely outward from the overlapped region by at least 0.5 inches.

36. The absorbent garment of claim 30, wherein the majority of the non-overlapped region of the polymeric film is elastomeric.

37. The absorbent garment of claim 30, wherein the entirety of the non-overlapped region of the polymeric film is elastomeric.

38. The absorbent garment of claim 30, wherein the laminate comprises two nonwoven layers superposed on opposing top and bottom surfaces of the polymeric film such that the polymeric film is sandwiched between the two nonwoven facings, wherein both the polymeric film and both nonwoven layers extend substantially through the entire area of the laminate.

39. The absorbent garment of claim 30, wherein the laminate defines a front laminate edge, and wherein an elastomeric waistband is attached to the front laminate edge and extends longitudinally between the front laminate edge and front edge.

40. The absorbent garment of claim 40, wherein the back region comprises an elastomeric film laminate that comprises an elastomeric film layer and a nonwoven layer, wherein both the elastomeric film layer and the nonwoven layer extend substantially throughout the entire area of the laminate in the back region;
wherein at least a portion of the absorbent member longitudinally overlaps at least a portion of the laminate in the back region to define in the laminate in the back region an overlapped region and a non-overlapped region;
wherein at least a portion of the overlapped region of the elastomeric film laminate in the back region is deactivated, and wherein at least a portion of the non-overlapped region of the elastomeric film laminate in the back region is elastomeric.

41. The absorbent garment of claim 40, wherein the elastomeric film laminate extends from the front region to the back region, such that the laminate is continuous and integral through the front region, crotch region, and back region.

42. The absorbent garment of claim 40, wherein the front region further defines a front crotch edge and the back region further defines a back crotch edge, such that the front region and the back region are indirectly connected to one another by way of the absorbent member.

43. An absorbent garment defining a longitudinal direction and a transverse direction, the absorbent garment comprising:
a front region defining a front edge, first and second transversely opposed front side edges, and a front crotch edge;
a back region defining a back edge, first and second transversely opposed back side edges, and a back crotch edge;
a crotch region disposed longitudinally between and interconnecting the front and back regions;
an absorbent member extending longitudinally from the front region to the back region, such that the front region and the back region are indirectly connected to one another by way of the absorbent member;
wherein the front region comprises an elastomeric film laminate that comprises an elastomeric film layer and a nonwoven layer, wherein both the elastomeric film layer and the nonwoven layer extend substantially throughout the entire area of the laminate in the front region;
wherein the back region comprises an elastomeric film laminate that comprises an elastomeric film layer and a nonwoven layer, wherein both the elastomeric film layer and the nonwoven layer extend substantially throughout the entire area of the laminate in the back region;
wherein at least a portion of the absorbent member longitudinally overlaps at least a portion of the laminate in front region to define in the laminate in the front region a front overlapped region and a front non-overlapped region;
wherein at least a portion of the absorbent member longitudinally overlaps at least a portion of the laminate in the back region to define in the laminate in the back region a back overlapped region and a back non-overlapped region;
wherein the entirety of the front overlapped region of the elastomeric film laminate is deactivated to define a front deactivated region, and wherein the majority of front non-overlapped region of the elastomeric film laminate is elastomeric.
wherein the entirety of the back overlapped region of the elastomeric film laminate is deactivated to define a back deactivated region, and wherein the majority of the back non-overlapped region of the elastomeric film laminate is elastomeric;
wherein the front deactivated region extends transversely outward from the front overlapped region by at least 0.5 inches on each side of the absorbent member, and wherein the back deactivated region extends transversely outward from the back overlapped region by at least 0.5 inches on each side of the absorbent member;
wherein the first front side edge is connected to the first back side edge and wherein the second front side edge is connected to the second back side edge, such that the garment is in a pull-on, pant-like configuration having a waist opening and two leg openings.

44. The absorbent garment of claim 43, wherein the front deactivated region extends longitudinally outward from the front overlapped region by at least 0.5 inches on each side of the absorbent member, and wherein the back deactivated region extends longitudinally outward from the back overlapped region by at least 0.5 inches on each side of the absorbent member;

45. The absorbent garment of claim 43, wherein the area of the front overlapped region at least 10% different than the area of the back overlapped region.

46. The absorbent garment of claim 45, wherein the area of the front overlapped region is at least 10% greater than the area of the back overlapped region.

47. The absorbent garment of claim 44, wherein the laminate of the front region defines a front laminate edge, and the laminate of the back region defines a back laminate edge, and wherein a front elastomeric waistband is attached to the front laminate edge and extends longitudinally between the front laminate edge and front edge, and wherein a back elastomeric waistband is attached to the back laminate edge and extends longitudinally between the back laminate edge and the back edge.
